(19) United States
(12) Patent Application Publication
(10) Pub. No.: US 2004/0067588 A1
(43) Pub. Date: Apr. 8, 2004

(54) Cell-free assay and in vivo method for plant genetic repair using chloroplast lysate

(76) Inventors: Gregory D. May, Ardmore, OK (US); Eric B. Kmiec, Landenberg, PA (US)

Correspondence Address: Eugenia S Hansen, Sidley Austin Brown & Wood, Suite 3400, 717 North Harwood Street, Dallas, TX, 75201, US

(21) Appl. No.: 10/250,632

(22) PCT Filed: Jan. 4, 2002
(86) PCT No.: PCT/US02/04583

(30) Foreign Application Priority Data
Jan. 5, 2001 (US) 60260076

(51) Int. Cl. A01H 1/00; C12N 15/82; C12N 15/74

(52) U.S. Cl. 435/468; 435/474

(57) ABSTRACT

An in vivo or in vitro cell-free method for genetic repair of mutation in plastid genes has been found which consists of (1) reacting a plasmid which contains a specific mutation (point mutation or frameshift mutation) of interest, a chimeric RNA/DNA oligonucleotide or a modified single stranded oligonucleotide which is believed to contain the genetic code for correcting the plastid gene mutation, and a chloroplast extract taken from the plant of interest, and (2) determining the success of gene conversion using a genetic readout system. A cell-free assay is disclosed by which the enzymatic capacity of chloroplast extracts to direct gene repair such as corrections to both point mutations and frameshift mutations can be determined. This assay method also enables the mechanistic study of plastid gene repair and facilitates the direct comparison between plant nuclear and organelle DNA repair pathways.

### TECHNICAL FIELD OF INVENTION

The invention relates to gene repair in plants.

### BACKGROUND OF THE INVENTION

Chimeric RNA/DNA (chimeras) and modified DNA oligonucleotides have be used to cause site-specific base changes in episomal and chromosomal targets in mammalian and plant cells (Kmiec, E. B. 1999. “Targeted gene repair,” Gene Therapy 6:1-4; May, G. D. and Kmiec, E. B. 2000. “Plant gene therapy: crop varietal improvement through the use of chimaeric RNA/DNA oligonucleotide-directed gene targeting,” AgBiotechNet 2:1-4, ABN 053; Beetham, et al. 1999. “A tool for functional plant genomics: chimeric RNA/DNA oligonucleotides cause in vivo gene-specific mutations,” Proc Natl Acad Sci USA 96: 8774-8778; Zhu, et al. 1999. “Targeted manipulation of maize genes in vivo using chimeric RNA/DNA oligonucleotides,” Proc Natl Acad Sci USA 96: 8768-8773; Zhu, et al. 2000. “Engineering herbicide-resistant maize using chimeric RNA/DNA oligonucleotides,” Nature Biotechnology 18:555-558; Rando, et al. 2000. “Rescue of dystrophin expression in mdx mouse muscle by RNA/DNA oligonucleotides,” Proc Natl Acad Sci USA 97:5363-5368 and cited references;). These molecules have been designed to pair with homologous sequences within target sites in genomic DNA and have been used to introduce a base change in previously characterized genomic DNA sequences.

Utilizing this approach, Rice et al (Rice, et al. 2000. “Genetic repair of mutations in plant cell-free extracts directed by specific chimeric oligonucleotides,” Plant Physiology 123:427-437) described the development of a cell-free nuclear extract system to study the mechanism of targeted gene correction in plants and as a tool to investigate plant DNA repair pathways. A plant cell-free nuclear extract obtained from monocots, dicots or embryonic tissue was used in conjunction with a chimeric RNA/DNA oligonucleotide or a modified DNA oligonucleotide to direct gene conversion of a plasmid which contained a gene with a point mutation or frameshift mutation in a biochemically controlled environment within a genetically tractable system.

The chloroplast genome (plastome) of eukaryotic algae and higher plants exists as closed circular molecules of double stranded DNA ranging from 80 to 200 kbp in size. Much of the chloroplast DNA (ctDNA) synthesis occurs in young leaf cells with copy numbers as high as 22,000 per cell during various stages of development. ctDNAs are redistributed to daughter organelles during plastid division. To maintain integrity of the plastome in mature leaves that are routinely subjected to high levels of UV irradiation, it is believed that efficient DNA repair pathways must exist in these organelles.

Chloroplast DNA homologous recombination and repair activities have been previously reported. The cloning of an Arabidopsis RecA protein with 53% identity to E. coli RecA (Cerutti, et al. 1992. “A homolog of Escherichia coli RecA protein in plastids of higher plants,” Proc Natl Acad Sci USA 89:8068-8072), has been reported as support for a possible endosymbiont relationship between chloroplast and other Eubacteria (Palmer, J. D. 1992. “Comparison of chloroplast and mitochondrial genome evolution in plants.” In Cell Organelles (Hermann, R. G., ed) Vienna: Springer-Verlag, pp. 99-133). Inhibition of ctDNA recombination and repair has been accomplished through the use of dominant negative mutants of E. coli RecA (Cerutti, et al. 1995. “Inhibition of chloroplast DNA recombination and repair by dominant negative mutants of Escherichia coli RecA,” Mol Cell Biol 15:3003-3011). Excision repair pathway enzyme activities have also been reported in chloroplasts of higher plants (Howland, et al. 1975. “Repair of DNA strand breaks after gamma-irradiation of protoplasts isolated from cultured wild carrot cells,” Mutation Res 1:81-87; and McLennan, A. G. 1988. “DNA damage, repair, and mutagenesis.” In DNA Replication in Plants; Bryant, J. A. and Dunham, V. L., eds., Boca Raton, Fla.: CRC Press, pp. 135-186).

Although the plastome encodes many of the proteins required for plastid function (Palmer, J. D. 1985. “Comparative organization of chloroplast genomes.” In Annual Review of Genetics; Campbell, A. Herskowitz, I. and Sandler, L. M., eds., Palo Alto, Calif.: Annual Reviews, Inc., pp. 325-354, for review), no DNA damage repair proteins have been reported to be encoded by the plastid genome (Britt, A. B. 1996. “DNA damage and repair in plants,” Ann Rev Plant Phys Plant Bio 47:75-100). Two Arabidopsis cDNAs that encode putative plastid targeting domains have been shown to complement an E. coli ruvC recG double mutant incapable of resolving cross-strand recombination intermediates (Pang, et al. 1993. “Two cDNAs from the plant Arabidopsis thaliana that partially restore recombination proficiency and DNA-damage resistance to E. coli mutant lacking recombination-intermediate-resolution activities,” Nucl Acids Res 21:1647-1653). These results indicate that a bioinformatics approach utilizing putative plastid targeting domains could be useful in sorting plant DNA recombination and repair enzymes, e.g., identifying proteins and homologues that are common or unique to plastid repair processes, or uncovering repair apparatus shared between the plastid and the nucleus.

We have now found an in vitro assay system and/or in vivo method that utilizes chimeric RNA/DNA oligonucleotides or modified DNA oligonucleotides in conjunction with chloroplast lysates for oligonucleotide-directed gene targeting. This assay provides a means by which plastid and genomic DNA repair activities can be evaluated and both plastid and nuclear oligonucleotide-directed repair and homologous recombination mechanisms can be studied.

### SUMMARY OF THE INVENTION

In one aspect, the invention is a method of modifying a target site of a plastid gene-of-interest comprising reacting an oligonucleotide that encodes a modification of the gene-of-interest, a duplex DNA molecule containing the gene-of-interest operably linked to a promoter so that the gene-of-interest can be expressed in a host organism, and a cell-free chloroplast lysate comprising components essential for recombination and gene repair activities and a mismatch repair activity, whereby the gene-of-interest is modified at the target site to form a modified gene-of-interest; introducing the modified gene-of-interest into the host organism; and detecting the expression of the modified gene-of-interest. In a preferred method, the oligonucleotide comprises at least 20 and less than or equal to 200 nucleotides. In another preferred method, the oligonucleotide comprises at least 10 and less than or equal to 100 Watson-Crick nucleotide pairs. In another preferred method, the oligonucleotide comprises a single 3′ end and a single 5′ end. The expression of the modified gene-of-interest can confer a selectable trait or an observable trait on the host organism.

In another aspect, the invention is a method of modifying a DNA sequence comprising reacting an oligonucleotide that encodes a modification of a DNA sequence, a duplex DNA molecule containing the DNA sequence, and a cell-free chloroplast lysate comprising components essential for recombination and gene repair activities and a mismatch repair activity to form a cell-free composition, whereby the DNA sequence is modified to form an altered DNA sequence, and detecting the altered DNA sequence. In one embodiment, the method further comprises fractionating the cell-free composition so as to enrich the altered DNA sequence relative to the DNA sequence, prior to detecting the altered DNA sequence. In a preferred method, the oligonucleotide comprises at least 20 and less than or equal to 200 nucleotides. In another preferred method, the oligonucleotide comprises at least 10 and less than or equal to 100 Watson-Crick nucleotide pairs. In another preferred method, the oligonucleotide comprises a single 3′ end and a single 5′ end. In another preferred method, the oligonucleotide is a duplex mutational vector comprising a contiguous single-stranded self-complementary oligonucleotide having a 3′ end and a 5′ end, wherein the 3′ end and the 5′ end are juxtaposed and wherein at least five contiguous nucleotides are Watson-Crick base paired, the sequence of the oligonucleotide comprising a template for the altered DNA sequence.

In another aspect, the invention is a cell-free composition for the modification of a DNA sequence comprising a duplex DNA containing a target sequence, an oligonucleotide which targets the DNA sequence and encodes the modification thereof, a cell-free chloroplast lysate comprising recombination and gene repair activities, and a reaction buffer. A preferred composition comprises an oligonucleotide comprising at least 20 and less than or equal to 200 nucleotides. Another preferred composition comprises an oligonucleotide comprising at least 10 and less than or equal to 100 Watson-Crick nucleotide pairs. Another preferred composition comprises an oligonucleotide comprising a single 3′ and a single 5′ end. Another preferred composition comprises a duplex DNA sequence which is a portion of a gene-of-interest that is operably linked to a promoter, so that the gene-of-interest can be expressed in a host organism. In one embodiment, the composition comprises a cell-free chloroplast lysate lacking mismatch repair activity. In this embodiment, the composition may comprise a cell-free chloroplast lysate which is a defined enzyme mixture of purified plant recombination and repair proteins capable of catalyzing plastid gene repair. The cell-free chloroplast lysate may be an extract of a plant cell, and the recombination and gene repair activities may be provided by a chloroplast-derived enzyme. In another embodiment, the composition comprises a cell-free chloroplast lysate which further comprises a mismatch repair activity. In this embodiment, the composition may comprise a cell-free chloroplast lysate which is a defined enzyme mixture of purified plant recombination and repair proteins capable of catalyzing plastid gene repair. The cell-free chloroplast lysate may be an extract of a plant cell, and the recombination and gene repair activities may be provided by a chloroplast-derived enzyme. A preferred composition comprises an oligonucleotide which is a duplex mutational vector comprising a contiguous single-stranded self-complementary oligonucleotide having a 3′ end and a 5′ end, wherein the 3′ end and the 5′ end are juxtaposed and wherein at least five contiguous nucleotides are Watson-Crick base paired, the sequence of the oligonucleotide comprising a template for the modified DNA sequence.

### BRIEF DESCRIPTION OF THE DRAWINGS

FIG. 1 depicts the targeted plasmids pK^{S}m4021 and pT^{S}Δ208, DNA targets and oligonucleotides used in the assays. Plasmids pK^{S}m4021 and pT^{S}Δ208 have been previously reported (Cole-Strauss, et al. 1999. “Targeted gene repair directed by the chimeric RNA/DNA oligonucleotide in mammalian cell-free extract,” Nucl Acids Res 27: 1323-1330; Gamper, et al.; and Rice, et al. 2000. Plant Physiology 123:427-437). As indicated, pK^{S}m4021 contains an intact ampicillin resistance gene and a mutated kanamycin gene; nucleotide 4021 is altered from a T to a G disabling kanamycin resistance. pT^{S}Δ208 has an intact wild-type amp^{r} gene and a mutated tetracycline gene; a frameshift mutation with a deleted C residue at nucleotide 208 disables tetracycline resistance. In the assay, chimeric oligonucleotide Kan4021C converts the point mutation in pK^{S}m4021 from a G to a C, re-establishing the capacity to confer kanamycin resistance in E. coli. Kan4021G is a control oligonucleotide that forms a perfect match with the target sequence in pKan^{s}m4021. Single-stranded vector 3S/25G converts the G residue to C in pK^{S}m4021. Chimeric oligonucleotide TetΔ208T is used to insert a T residue at position 208, as does single-strand vector 3S/28A. SC1 is a nonspecific chimeric oligonucleotide (see Cole-Strauss, et al. 1996. “Correction of the mutation responsible for sickle cell anemia directed by an RNA/DNA oligonucleotide,” Science 273: 1386-1389) bearing no sequence complementarity to the target site. The highlighted base (inside square) illustrates the position within the oligonucleotide that mismatches with the target sequence. The asterisks between bases in 3S/25G and 3S/28A, respectively, indicate the positions of the phosphothioate linkages.

FIG. 2 depicts the DNA sequence of the converted pK^{s}m4021 plasmids. Confirmation of sequence alteration in isolated plasmid, directed by the indicated chimeric oligonucleotide (CO) or the indicated modified single-stranded oligonucleotide (MO) is displayed. This represents a repair of a point mutation (G→C) and the altered residues are found at the following positions as numbered in the sequences: Control, position 89; CO/Pre-Ext, position 86; MO/Pre-Ext, position 89; CO/Post-Ext, position 86; and MO/Post-Ext, position 89.

FIG. 3 depicts the DNA sequence of converted pT^{s}Δ208 plasmids. Confirmation of sequence alteration in isolated plasmid DNA, directed by the indicated chimeric oligonucleotide (CO) or indicated modified single-stranded oligonucleotide (MO) is displayed. The correction involves the repair of a frameshift mutation (T insertion) at position 167 for CO and position 166 for MO.

### DETAILED DESCRIPTION

We have now found a method by which chloroplast extracts can be used in conjunction with a chimeric RNA/DNA oligonucleotide or a modified single stranded oligonucleotide to direct plastid or nuclear gene conversions, e.g., correction of point mutations or frameshift mutations.

In one aspect, the present invention is a cell-free assay in which gene conversion is conducted in a biochemically controlled environment within a genetically tractable system. The cell-free assay is useful for elucidating plastid DNA recombination and repair pathways in plant cells as well as the identification and characterization of proteins involved in the process. The demonstration that the chloroplast extract supports the correction of a point mutation and/or frameshift mutation in the assay indicates that the chloroplasts possess the machinery to catalyze correction of either one or both types of mutations. Furthermore, the cell-free assay of the present invention provides a means by which to compare DNA repair pathways that maintain the integrity of the plastid and nuclear genomes, and provide tools to elucidate both plastid and nuclear oligonucleotide-directed gene conversion and homologous recombination mechanisms. In another aspect, the present invention is a method by which plastid gene conversion is conducted in vivo.

The cell-free assay of the present invention provides a method by which a chloroplast extract from a plant of interest is screened for its ability to support point mutation or frameshift mutation gene conversion. In general, the cell-free assay consists of (1) an in vitro reaction involving a plasmid which contains a specific mutation (point mutation or frameshift mutation) of interest, a chimeric RNA/DNA oligonucleotide or a modified single stranded oligonucleotide which is believed to contain the genetic code for correcting the gene mutation of interest in the plasmid, and a chloroplast extract taken from the plant of interest; and (2) a genetic readout system for determining gene conversion, e.g., the mutated gene conferring antibiotic resistance, as wild-type, when introduced into E. coli followed by quantitation of plasmid repair events by plating the bacteria on agarose laden with the appropriate antibiotic.

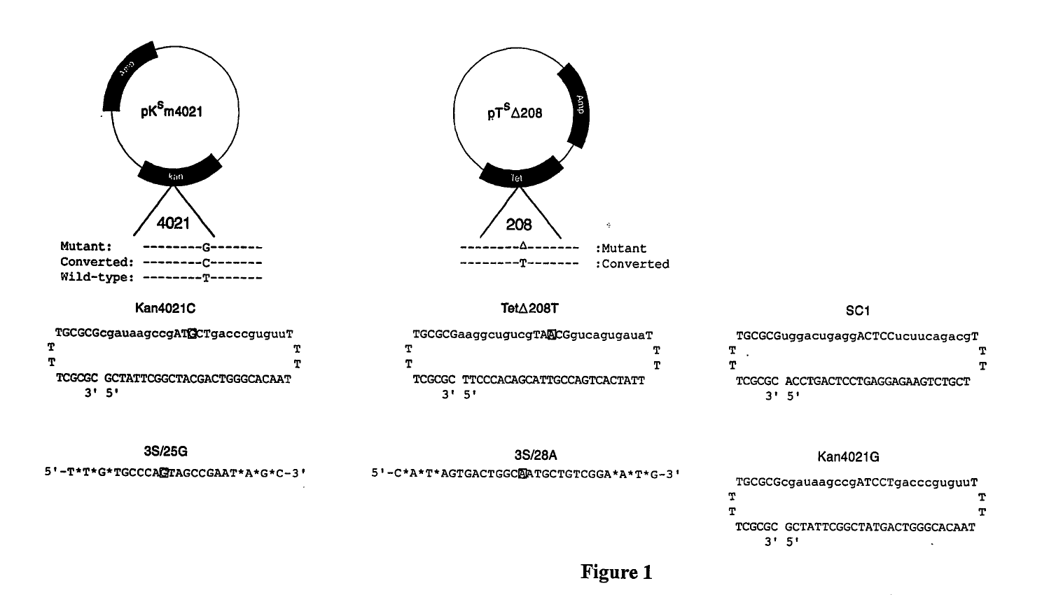

To detect gene correction, it is believed that any system known in the art which identifies the correction of point or frameshift mutations in a cell-free environment can be used. Preferably, a system using plasmid molecules containing point or frameshift mutations in the coding regions of antibiotic resistance genes is used. Plasmids used in the exemplary model systems shown herein are pK^{S}m4021 and pT^{S}Δ208. As shown in FIG. 1, plasmid pK^{S}m4021 contains a point mutation at nucleotide 4021 located with coding region of the kan^{r} gene wherein the wild type, T (thymine), has been changed to G (guanine), and a wild-type ampicillin resistance gene. As indicated in FIG. 1, the chimera used in the assay of the present invention converts the G (guanine) at position 4021 to C (cytosine), instead of T (thymine). This switch for replacing G (guanine) with C (cytosine) rather than the wild type T (thymine) allows the generation of a functional protein that preserves the phenotypic readout as kanamycin resistance while ensuring that kanamycin resistance has developed through conversion directed by the oligonucleotide and not through wild-type plasmid contamination. Plasmid pT^{S}Δ208 contains a frameshift mutation in which a C (cytosine) residue at position 208 has been removed, rendering the plasmid incapable of providing tetracycline resistance, and a wild-type ampicillin resistance gene. As indicated in FIG. 1, the chimera TetΔ208T used in the assay of the present invention inserts a T (thymine) residue rather than a C (cytosine) at position 208. This switch for inserting T (thymine) rather than C (cytosine) allows the generation of a functional protein that preserves the phenotypic readout as tetracycline resistance while ensuring that tetracycline resistance has developed through conversion directed by the oligonucleotide and not through wild-type plasmid contamination. The presence of the ampicillin gene in the plasmids enables control and normalization of the transfection process.

To detect gene correction, it is believed that any type of oligonucleotide known in the art which is capable of correcting point or frameshift mutations in a cell-free environment can be used. Two basic types of oligonucleotides providing for the correction of point or frameshift mutations in a cell-free environment are preferably used in the present invention (Gamper, et al. 2000. Biochem 39:5808-5816; and Gamper, et al. 2000. “The DNA strand of chimeric RNA/DNA oligonucleotides can direct gene repair/conversion activity in mammalian and plant cell-free extracts,” Nucl Acids Res 28:4332-4339). One type is a chimeric RNA/DNA oligonucleotide (CO) which consists of complementary RNA and DNA residues folded into a double hairpin configuration resistant to cellular nucleases due to the 4T (thymine) residues at each hairpin end, comprising at least 10 and less than or equal to 100 Watson-Crick nucleotide pairs with a single 3′ end and a single 5′ end. FIG. 1 shows an exemplary CO, Kan4021C, used in the conversion of plasmid pK^{S}m4021 and TetΔ208T used in the conversion of plasmid pT^{S}Δ208. The second type is a modified single stranded oligonucleotide (MO) comprising at least 20 and less than or equal to 200 nucleotides, more preferably a 25-mer consisting of all DNA residues but having phosphothioate linkages between the terminal four bases on each end. FIG. 1 gives two exemplary MO, 3S/25G used in the conversion of pK^{S}m4021 and 3S/28A for the conversion of pT^{S}Δ208. These molecules are also resistant to nuclease digestion in the cell-free extract. wherein said oligonucleotide comprises at least 20 and less than or equal to 200 nucleotides with a single 3′ end and a single 5′ end.

In the assay of the present invention, a chloroplast extract from a plant of interest is screened for its ability to support point mutation or frameshift mutation gene conversion. Any chloroplast lysate preparation method which maintains the integrity of the organelle's machinery to catalyze the correction of either point mutations or frameshift mutations or both can be used in the present invention. Two standard methods of mechanically preparing chloroplast extracts useful in the present invention are presented herein. The first, a “pre-gradient” preparation, is obtained by gentle resuspension of the pelleted chloroplasts following low-speed centrifugation. The second, a “post-gradient” preparation is obtained by using a simple Percoll step gradient.

In the assay of the present invention, any genetic readout system capable of demonstrating a gene conversion of the selected point or frameshift mutation can be used. For example, successful gene conversion of plasmid molecules containing a point or frameshift mutation in the coding region of an antibiotic resistance gene can be detected by introducing the plasmid into a bacteria normally sensitive to the antibiotic, plating the transformed bacteria on agarose laden with the appropriate antibiotic, and examining the agarose culture for detectable bacterial colonies, wherein each bacterial colony represents at least one plasmid repair event from antibiotic sensitivity to antibiotic resistance.

In a preferred assay of the present invention, a chloroplast extract of interest is mixed with a plasmid having a point mutation or a frameshift mutation in an target gene such as an antibiotic resistant gene and an oligonucleotide designed to correct the error. After the initial reaction mixture is incubated under conditions to promote gene conversion (e.g., at about 37° C. for about one hour), the plasmids are isolated and transformed by any means known in the art (e.g., electroporated) into competent Escherichia coli cells harboring a mutation in the recA gene. E. coli strain DH10B is a preferred strain deficient in RecA activity which is known to participate in recombination events in E. coli. Based on previous work confirming that the repair reaction takes place in cell-free extract from plants (Rice, et al. 2000. Plant Physiology 123:427-437), the use of cells deficient in RecA function ensures that any correction observed after the phenotypic readout occurs in the cell-free extract. The correction events are scored by selection on agar plates containing the target antibiotic. Preferably, dilutions from the same transformation are plated in duplicate and selected on plates containing ampicillin to normalize the efficiency of electroporation. Frequencies are calculated as target antibiotic revertant colonies relative to ampicillin resistant colonies selected from the same reaction sample. Since, the plasmids also have an intact copy of an ampicillin resistance gene, colonies arising on the target antibiotic plates should be resistant to ampicillin. In addition, the ampicillin colonies provide a way to normalize potential variations in colony counts due to the transformation process.

The cell-free assay of the present invention will be more clearly understood with reference to the exemplary model systems described as follows.

### EXAMPLE 1

### Oligonucleotide-Directed Gene Repair Assay Correcting Point Mutations and Frameshift Mutations

The ability of chloroplast preparations to support the correction of a point mutation in Plasmid pK^{S}m4021 was examined.

Plant materials

Spinach (cv. Trias) was grown in a growth chamber from seed in MetroMix 350 for four weeks under 12 hour, 20′ days.

Preparation of Chloroplast Lysates

Chloroplasts were mechanically isolated based on previously published methods (Whitehouse, D. G. and Moore, A. L. 1993. “Isolation and purification of fictionally intact chloroplasts from leaf tissue and leaf tissue protoplasts.” In Methods in Molecular Biology, Vol. 19: Biomembrane Protocols: I. Isolation and Analysis; Graham, J. M. and Higgins, J. A., eds., Totowan, N.J.: Humana Press, Inc., pp.123-151). Briefly, 50 grams of freshly harvested young spinach leaves were rinsed in ice-cold water, blotted dry to remove excess water, and deribbed. Leaf materials were finely sliced, placed in a chilled beaker containing 150 milliliters of ice-cold isolation medium (330 mM sorbitol, 10 mM Na₂P₄O₇, 5 mM MgCl₂, and 2 mM Na-isoascorbate adjusted to pH 6.5 with HCl), and disrupted into a slurry using short bursts of a Polytron tissue homogenizer (Brinkmann Instruments, Inc., Westbury, N.Y.). The resulting slurry was squeezed first through two layers of muslin and subsequently passed through a muslin cotton wool sandwich into a 250 ml beaker on ice. The filtrate was divided equally, centrifuged for 1 min at 3000 g, the supernatants were decanted, and the pellets resuspended in 1.0 milliliter of resuspension medium (330 mM sorbitol, 50 mM HEPES-KOH pH 7.6, 2 mM EDTA, 1 mM MgCl₂ and 1 mM MnCl₂). Samples were washed in a total 150 milliliters of resuspension medium, and centrifuged as above. To enhance the percentage of intact chloroplasts, one half of the sample was gently resuspended in 1 milliliter of ice-cold resuspension medium and layered onto a 6 ml cushion of 40% (v/v) Percoll containing osmoticum/buffer and centrifuged at 3000 g for 1 min. The pellets of the “pre-” and “post-gradient” samples were lysed in 300 microliters of lysate buffer (20 mM HEPES, pH 7.5, 5 mM KCl, 1.5 mM MgCl₂, 10 mM DTT, 10% [v/v] glycerol, and 1% [w/v] PVP). Samples were then homogenized with 20 strokes of a Dounce homogenizer (Bellco Glass, Inc., Vineland, N.J.). Following homogenization, samples were incubated on ice for 1 hour and centrifuged at 3000 g for 5 min to remove debris. Protein concentrations of the supernatants were determined by Bradford assay (Bradford, M. M. 1976. “A rapid and sensitive method for the quantitation of microgram quantities of protein utilizing the principle of protein-dye binding,” Anal Biochem 72:248-254). Extracts were dispensed into 100 microgram aliquots, frozen in a dry ice-ethanol bath and stored at −80° C.

Plasmids

Kanamycin and tetracycline resistance genes were used in two substitutory systems to determine nucleotide exchange in chloroplast lysates. The kanamycin-sensitive plasmid pK^{s}m4021 containing a single base transversion (T→G), that creates a TAG stop codon in the kan gene at codon 22 was used to illustrate correction of a point mutation in chloroplast lysates. A nucleotide insertional system with a tetracycline sensitive plasmid, pT^{S}Δ208, was used to analyze repair of single base deletions in chloroplast lysates. The plasmid carries a single nucleotide deletion at position 208, which creates a frameshift in the tet gene of pBR322 at codon 41. The plasmids also contained a wild-type ampicillin gene used for propagation and normalization (Cole-Strauss, et al. 1999. Nucl Acids Res 27: 1323-1330).

Oligonucleotides

Synthetic oligonucleotides were used to direct reversion of kan^{S} and tet^{S} genes to restore resistance to their respective antibiotics. The chimeric RNA/DNA oligonucleotide Kan4021C, which can direct conversion of the kan^{S} gene in pK^{S}m4021 at codon 22 from TAG to TAC (stop→tyrosine), was synthesized as previously described (Cole-Strauss, et al. 1999. Nucl Acids Res 27: 1323-1330). Chimeric RNA/DNA oligonucleotide TetΔ208T was used to revert the tet^{S} gene of plasmid pT^{S}Δ208, at the mutated base. A non-specific chimera SC1 (Cole-Strauss, et al. 1996. Science 273: 1386-1389) was used for comparison and as a control. Single stranded oligonucleotides 3S/25G and 3S/28A were synthesized with the appropriate modifications using phosphoramidites or controlled pore glass supports. After deprotection and removal from the solid support, all oligonucleotides were gel-purified according to Gamper et al (Gamper, et al. 2000. Biochem 39:5808-5816; and Gamper, et al. 2000. Nucl Acids Res 28:4332-4339) and concentrations determined spectrophotometrically (33 or 44 micrograms/milliliter per A₂₆₀ unit).

In Vitro Assays

Reaction mixtures consisted of 1 microgram of substrate plasmid pK^{S}m4021 and 1.5 micrograms of either chimeric oligonucleotide Kan4021 C and the nonspecific CO SCI, or 0.55 to 1.5 micrograms of modified oligonucleotide Kan 3S/25G for the kan^{S} system. For the tet^{S} system, 1 microgram of substrate plasmid pT^{S}Δ208 and 1.5 micrograms of effector oligonucleotide TetΔ208T or 0.55 micrograms of the modified oligonucleotide 3S/28A were used. These components were mixed in a buffer of 200 mM Tris, pH 7.5, 100 mM MgCl₂, 1 mM DTT, 0.2 mM spermidine, 25 mM ATP, 1 mM each CTP, GTP, UTP, 0.1 mM each dNTPs, and 10 mM NAD. The reaction was initialized by adding 0 to 20 micrograms chloroplast lysates in 100 microliter reaction volumes. The reactions were incubated at 30° C. for 30 min and stopped by placing on ice. The substrate plasmid was then isolated by phase partition with 1:1 phenol:chloroform extraction, followed by ethanol precipitation on dry ice for 2 hours or overnight and centrifugation at 4° C. for 30 min. Samples were then washed with 70% ethanol and centrifuged for 15 min and resuspended in 50 microliters TE.

Electroporation, Plating and Selection

For the E. coli transformation, 5 microliters of resuspended reaction precipitates were used to transform 20 microliter aliquots of electrocompetent E. coli DH10B using a Cell-Porator apparatus (Life Technologies, Inc., Rockville, Md.) as described by the manufacturer. Each mixture was transferred to a 1 milliliter SOC culture, incubated at 37° C. for 1 hour, and then converted plasmids were amplified by adding kanamycin to 50 micrograms/milliliter or tetracycline to 12 micrograms/milliliter and an additional incubation for 3 hours at 37° C. Then, 100 microliter aliquots of undiluted cultures were plated onto LB agar plates containing 50 micrograms/microliter kanamycin or 12 micrograms/milliliter tetracycline, respectively. Also, 100 microliter aliquots of a 10⁴ dilution of the cultures were plated onto LB agar plates containing 100 milligrams/milliliter ampicillin. Plating was performed in duplicate using sterile Pyrex beads. Both sets of plates were incubated for 16 to 18 hours at 37° C., and colonies were counted using an Accucount 1000 plate reader (BioLogics, Inc., Gainesville, Va.). Targeted conversion of the kan^{S} or tet^{S} gene was determined by normalizing the number of kanamycin resistant or tetracycline resistant colonies by dividing by the number of ampicillin resistant colonies, since all plasmids contain a wild type amp gene. Resistant colonies were confirmed by selecting isolated clones for mini preparation of plasmid DNA followed by sequencing using an ABI Big Dye Terminator on an automated ABI 310 capillary sequencer (Applied Biosystems, Foster City, Calif.).

Correction of Point Mutation

As shown in Table I, both the pre-gradient and post-gradient chloroplast extracts promoted gene repair of plasmid pKan^{s}m4021. This table presents the average colony count of five independent reactions for each type of extract and at varying levels. Furthermore, a direct comparison between the chimeric oligonucleotide and the single-stranded vector, 3S/25G, is shown in Table I. In all four sets of reactions, a dose-dependent response of chloroplast extract was found, but a maximal number of colonies was generated using 10-20 micrograms of extract. The more purified post-gradient chloroplast extract supported a higher level of repair, and 3S/25G was more efficient in directing the conversion reaction.

Table II illustrates that all reaction components had to be present for antibiotic-resistant colonies to arise. Spurious colonies were occasionally found in some of the control plates. However, upon sequencing, these few colonies did not harbor the corrected, targeted base, suggesting that they might be due to random reversion.

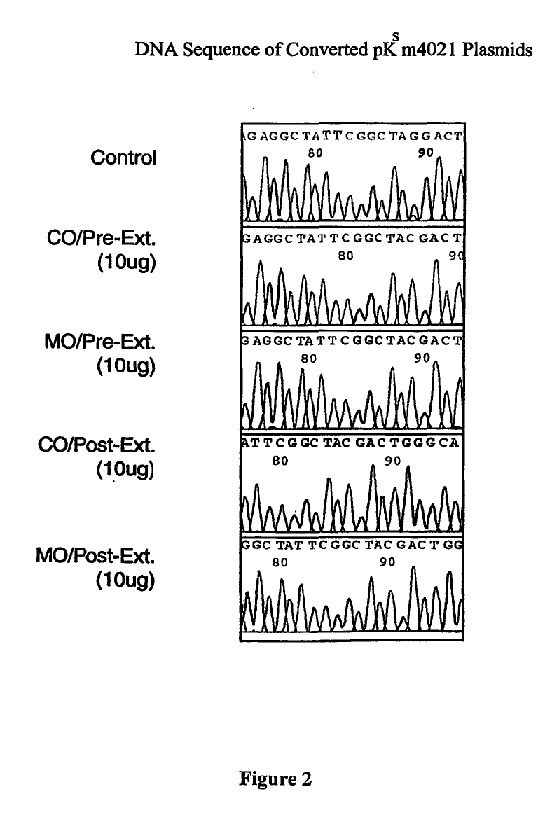

Plasmid DNA harbored in three colonies from each reaction point was isolated and processed for DNA sequencing. As shown in FIG. 2, chimeric oligonucleotides and single-stranded vectors directed precise targeted gene repair. While only five sequencing reactions are shown, all samples produced the same result. In addition, the complementary strand of the repaired plasmid target was sequenced and found to contain the proper complementary base at the correct position (data not shown).

Since the post-gradient extract was more highly purified and likely to more closely reflect the contents of the chloroplast fraction, this source of extract was used to determine the optimal dosage of oligonucleotide. Because the single-stranded, 3S/25G, oligonucleotide is approximately 50% smaller than the chimeric oligonucleotide (70 nucleotides), in terms of molecules, a unit amount (microgram) of MO would contain more correction vehicles than the same amount of CO. Thus, the dose curve was adjusted so that approximately the same numbers of molecules were present in each reaction. The results, shown in Table III, displayed a dose-dependency for both vectors, and confirmed that MOs were more efficient in directing gene repair even when the number of correction vehicles were the same. Finally, oligonucleotides that either form a perfect match (Kan4021G) or are nonspecific (SC1) for the target site were tested. No antibiotic-resistant colonies were generated at several different dosages.

Correction of Frameshift Mutation

Plasmid pT^{S}Δ208 (FIG. 1) contains a frameshift mutation at position 208 in the coding region of the tetracycline resistance gene. This plasmid was mixed with the appropriate oligonucleotides, and the reaction was initialized by the addition of the post-gradient chloroplast extract. As shown in Table IV (reactions 1-10), correction of the frameshift mutation was enabled by the extract and either CO (TetΔ208T) or MO (3S/28A). The colony number was reduced when compared to the numbers found when a point mutation was targeted for repair. The level of correction was dependent on the amount of extract added, and the number of colonies was higher when the single-stranded vector was used. The difference in repair efficiency between the two types of vectors in this case, however, was modest; this may reflect the difficulty in repairing a frameshift mutation as opposed to a point mutation since each type of event requires different members of the repair protein family.

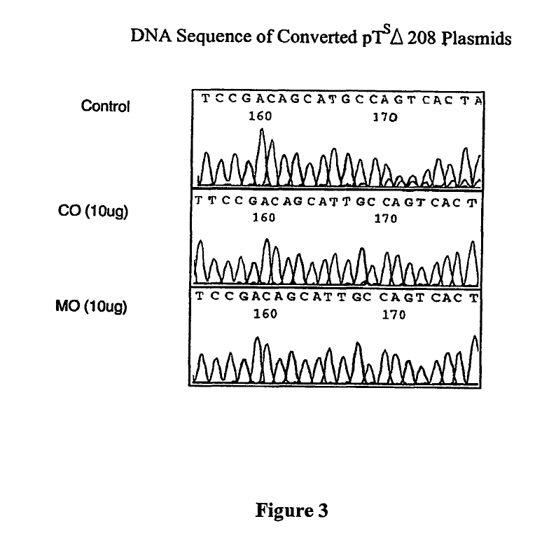

Three colonies from each set of reactions were selected and the plasmid DNA sequenced around the target site. FIG. 3 illustrates a representative sequence from each set, and the specified nucleotide (T) has been inserted at the targeted location.

The assay of the present invention can be used to readily assess whether the chloroplasts in a given plant or plant tissue has sufficient enzymatic machinery to catalyze the reactions necessary for gene conversion. The assay system of the present invention provides a means by which chloroplast supported gene conversion mechanisms can be elucidated and monitored. The assay of the present invention can also be used to demonstrate what types of DNA repair proteins are present in chloroplasts from a selected plant tissue. This assay system provides a means by which such proteins and eventually their genes can be isolated. The cell-free extract can be fractionated, and biochemical purification of the active proteins can be enabled. For any purification protocol, the single most important aspect is a reliable assay system to follow the activity. The chloroplast cell-free extract provides such a test system.

The assay of the present invention can be used to determine if environmental stimuli increase the efficiency of chimeras in plant cells, i.e., if exposure of plants or plant cells to chemical mutagens, UV, gamma, or other high energy sources stimulate chloroplast machinery resulting in a corresponding increase in chimera efficiency. Likewise, the molecular components associated with the response to environmental stimuli can be identified.

The assay of the present invention provides a means to compare DNA repair pathways that maintain the integrity of the plastid and nuclear genomes. Since no DNA damage repair proteins have been reported to be encoded by the plastid genome (Britt, A. B. 1996. Ann Rev Plant Phys Plant Bio 47:75-100), targeting domains can identify which nuclear encoded DNA repair proteins are destined to the plastid. The ability to compare different and physically separate DNA repair pathways between organelles within the same cell elucidates factors effecting fundamental differences in homologous and illegitimate recombination mechanisms observed between plastid and nuclear genomes.

In vivo modification of a plastid gene-of-interest can be accomplished by: 1) providing an oligonucleotide that encodes a modification of the gene-of-interest, providing a duplex DNA molecule containing the gene-of-interest operably linked to a promoter so that the gene-of-interest can be expressed in a host organism, providing a cell-free chloroplast lysate comprising recombination and gene repair activities and a mismatch repair activity, 2) reacting the oligonucleotide, duplex DNA molecule, and cell-free chloroplast lysate whereby the gene-of-interest is modified at the target site to form a modified gene-of-interest; and 3) introducing the modified gene-of-interest into the host organism. To detect the expression of the modified gene-of-interest, a selectable marker trait or an observable trait can be utilized.

TABLE I
Oligonucleotide-directed gene repair in chloroplast extracts^{a}

| Plasmid | Chimeric oligo (CO) | Pre-Ex. (μg) | Post-Ex. (μg) | No. observed |
|---|---|---|---|---|
| 1. pK^{S}m4021(1 μg) | Kan 4021C(1.5 μg) | 2.5 | — | 61 |
| 2. pK^{S}m4021(1 μg) | Kan 4021C(1.5 μg) | 5 | — | 135 |
| 3. pK^{S}m4021(1 μg) | Kan 4021C(1.5 μg) | 10 | — | 199 |
| 4. pK^{S}m4021(1 μg) | Kan 4021C(1.5 μg) | 20 | — | 235 |
| 5. pK^{S}m4021(1 μg) | Kan 4021C(1.5 μg) | — | 2.5 | 53 |
| 6. pK^{S}m4021(1 μg) | Kan 4021C(1.5 μg) | — | 5 | 103 |
| 7. pK^{S}m4021(1 μg) | Kan 4021C(1.5 μg) | — | 10 | 191 |
| 8. pK^{S}m4021(1 μg) | Kan 4021C(1.5 μg) | — | 20 | 273 |
| Plasmid | Modified oligo (MO) | Pre-Ex. (μg) | Post-Ex. (μg) | No. observed |
| 11. pK^{S}m4021(1 μg) | Kan 3S/25G(1.5 μg) | 2.5 | — | 77 |
| 12. pK^{S}m4021(1 μg) | Kan 3S/25G(1.5 μg) | 5 | — | 123 |
| 13. pK^{S}m4021(1 μg) | Kan 3S/25G(1.5 μg) | 10 | — | 229 |
| 14. pK^{S}m4021(1 μg) | Kan 3S/25G(1.5 μg) | 20 | — | 315 |
| 15. pK^{S}m4021(1 μg) | Kan 3S/25G(1.5 μg) | — | 2.5 | 94 |
| 16. pK^{S}m4021(1 μg) | Kan 3S/25G(1.5 μg) | — | 5 | 379 |
| 17. pK^{S}m4021(1 μg) | Kan 3S/25G(1.5 μg) | — | 10 | 584 |
| 18. pK^{S}m4021(1 μg) | Kan 3S/25G(1.5 μg) | — | 20 | 612 |
| ^{a}Each reaction contained 1.0 μg of plasmid DNA and 1.5 μg of oligonucleotide mixed with the indicated amounts of extract. Genetic readout took place in DH10B (recA1), and colony counts reflect the average of five independent experiments. Variations among samples was less than 15%. Kanamycin resistant colonies are per | | | | |
| #10⁷ ampicillin resistant colonies that were quantified by duplicate plating. | | | | |

TABLE II
Gene correction requires reaction components^{a}

| Plasmid (1 μg) | Chimeric oligo (1.5 μg) | Pre-Ex. (μg) | Post-Ex. (μg) | No. observed |
|---|---|---|---|---|
| 1. pKan^{S}m4021 | Kan4021C | — | — | 5 |
| 2. pKan^{S}m4021 | — | 10 | — | 2 |
| 3. pKan^{S}m4021 | — | — | 10 | 2 |
| 4. pKan^{S}m4021 | — | — | — | 1 |
| 5. pKan^{S}m4021 | Kan4021C | 10 | — | 123 |
| 6. pKan^{S}m4021 | Kan4021C | — | 10 | 258 |
| Plasmid (1 μg) | Modified oligo (1.5 μg) | Pre-Ex. (μg) | Post-Ex. (μg) | No. observed |
| 11. pKan^{S}m0421 | 3T/25G | — | — | 1 |
| 12. pKan^{S}m4021 | — | 10 | — | 1 |
| 13. pKan^{S}m4021 | — | — | 10 | 0 |
| 14. pKan^{S}m4021 | — | — | — | 6 |
| 15. pKan^{S}m4021 | 3S/25G | 10 | — | 141 |
| 16. pKan^{S}m4021 | 3S/25G | — | 10 | 437 |

^{a}The reaction mixture contained the indicated components. Plasmid DNA was electroporated into DH10B cells and colony counts determined by antibiotic resistance. The results represent an average of five independent reactions.

TABLE III
Dosage dependence of gene repair^{a}

| Plasmid | CO (μg) | MO (μg) | Post-Extract (μg) | No. observed |
|---|---|---|---|---|
| 1. pKan^{S}m4021 | 3.0 | — | — | 1 |
| 2. pKan^{S}m4021 | — | 3.0 | — | 0 |
| 3. pKan^{S}m4021 | 0.25 | — | 10 | 30 |
| 4. pKan^{S}m4021 | 0.5 | — | 10 | 47 |
| 5. pKan^{S}m4021 | 1.5 | — | 10 | 116 |
| 6. pKan^{S}m4021 | 3.0 | — | 10 | 271 |
| 7. pKan^{S}m4021 | — | 0.08 | 10 | 81 |
| 8. pKan^{S}m4021 | — | 0.175 | 10 | 212 |
| 9. pKan^{S}m4021 | — | 0.52 | 10 | 317 |
| 10. pKan^{S}m4021 | — | 1.05 | 10 | 399 |
| 11. pKan^{S}m4021 | Kan4021G(0.5) | — | 10 | 0 |
| 12. pKan^{S}m4021 | SCI(0.5) | — | 10 | 0 |
| 13. pKan^{S}m4021 | Kan4021G(3.0) | — | 10 | 0 |
| 14. pKan^{S}m4021 | SCI(3.0) | — | 10 | 0 |
| ^{a}Reactions contained plasmid (1 μg) and oligonucleotides at the indicated amounts and were initialized by addition of 10 μg of post-extract. The number of kan^{r} colonies were determined after electroporation in DH10B E. coli and counting on kanamycin plates. Kan^{r} colonies are per 10⁷ amp^{r} colonies. | | | | |
| #The colony numbers represent three independent reactions. | | | | |

TABLE IV
Gene repair of frameshift and point mutations in a mutated tet^{r} gene^{a}

| Plasmid (1 μg) | CO (1.5 μg) | MO (0.52 μg) | Post-Extract (μg) | No. Observed |
|---|---|---|---|---|
| 1. pT^{S}Δ208 | TetΔ208T | — | — | 0 |
| 2. pT^{S}Δ208 | — | 3S/28A | — | 0 |
| 3. pT^{S}Δ208 | TetΔ208T | — | 2.5 | 13 |
| 4. pT^{S}Δ208 | TetΔ208T | — | 5 | 29 |
| 5. pT^{S}Δ208 | TetΔ208T | — | 10 | 42 |
| 6. pT^{S}Δ208 | TetΔ208T | — | 20 | 71 |
| 7. pT^{S}Δ208 | — | 3S/28A | 2.5 | 19 |
| 8. pT^{S}Δ208 | — | 3S/28A | 5 | 37 |
| 9. pT^{S}Δ208 | — | 3S/28A | 10 | 59 |
| 10. pT^{S}Δ208 | — | 3S/28A | 20 | 93 |
| ^{a}Reactions contained the indicated components with varying amounts of extract. Reactions 1-10 contained pT^{S}Δ208 and the appropriate oligonucleotide. Colony counts were determined by genetic readout in E. coli (DH10B). The number of kan^{r} colonies are per 10⁷ amp^{r} colonies and represent the average | | | | |
| #of three independent reactions. | | | | |

We claim:

1. A method of modifying a target site of a plastid gene-of-interest comprising:
reacting an oligonucleotide that encodes a modification of said gene-of-interest, a duplex DNA molecule containing said gene-of-interest operably linked to a promoter so that said gene-of-interest can be expressed in a host organism, and a cell-free chloroplast lysate comprising components essential for recombination and gene repair activities and a mismatch repair activity, whereby said gene-of-interest is modified at said target site to form a modified gene-of-interest;
introducing said modified gene-of-interest into said host organism; and
detecting the expression of said modified gene-of-interest.

2. The method of claim 1, wherein said oligonucleotide comprises at least 20 and less than or equal to 200 nucleotides.

3. The method of claim 1, wherein said oligonucleotide comprises at least 10 and less than or equal to 100 Watson-Crick nucleotide pairs.

4. The method of claim 1, wherein said oligonucleotide comprises a single 3′ end and a single 5′ end.

5. The method of claim 1, 2, 3 or 4, wherein said expression of said modified gene-of-interest confers a selectable trait on said organism.

6. The method of claim 1, 2, 3 or 4, wherein said expression of said modified gene-of-interest confers an observable trait on said organism.

7. A method of modifying a DNA sequence comprising:
reacting an oligonucleotide that encodes a modification of a DNA sequence, a duplex DNA molecule containing said DNA sequence, and a cell-free chloroplast lysate comprising components essential for recombination and gene repair activities and a mismatch repair activity to form a cell-free composition, whereby said DNA sequence is modified to form an altered DNA sequence, and
detecting said altered DNA sequence.

8. The method of claim 7, further comprising fractionating said cell-free composition so as to enrich said altered DNA sequence relative to said DNA sequence, prior to detecting said altered DNA sequence.

9. The method of claim 7 or 8, wherein said oligonucleotide comprises at least 20 and less than or equal to 200 nucleotides.

10. The method of claim 7 or 8, wherein said oligonucleotide comprises at least 10 and less than or equal to 100 Watson-Crick nucleotide pairs.

11. The method of claim 7 or 8, wherein said oligonucleotide comprises a single 3′ end and a single 5′ end.

12. The method of claim 7 or 8, wherein said oligonucleotide is a duplex mutational vector comprising a contiguous single-stranded self-complementary oligonucleotide having a 3′end and a 5′end, wherein said 3′ end and said 5′end are juxtaposed and wherein at least five contiguous nucleotides are Watson-Crick base paired, the sequence of said oligonucleotide comprising a template for said modified DNA sequence.

13. A cell-free composition for the modification of a DNA sequence comprising a duplex DNA containing a target sequence, an oligonucleotide which targets the DNA sequence and encodes the modification thereof, a cell-free chloroplast lysate comprising recombination and gene repair activities, and a reaction buffer.

14. The composition of claim 13, wherein said oligonucleotide comprises at least 20 and less than or equal to 200 nucleotides.

15. The composition of claim 13, wherein said oligonucleotide comprises at least 10 and less than or equal to 100 Watson-Crick nucleotide pairs.

16. The composition of claim 13, wherein said oligonucleotide comprises a single 3′ and a single 5′ end.

17. The composition of claim 13, wherein said duplex DNA sequence is a portion of a gene-of-interest that is operably linked to a promoter, so that said gene-of-interest can be expressed in a host organism.

18. The composition of claim 13, wherein said cell-free chloroplast lysate lacks mismatch repair activity.

19. The composition of claim 18, wherein said cell-free chloroplast lysate is a defined enzyme mixture of purified plant recombination and repair proteins capable of catalyzing plastid gene repair.

20. The composition of claim 19, wherein said cell-free chloroplast lysate is an extract of a plant cell.

21. The composition of claim 19, wherein said recombination and gene repair activities are provided by a chloroplast-derived enzyme.

22. The composition of claim 13, wherein said cell-free chloroplast lysate further comprises a mismatch repair activity.

23. The composition of claim 22, wherein said cell-free chloroplast lysate is a defined enzyme mixture of purified plant recombination and repair proteins capable of catalyzing plastid gene repair.

24. The composition of claim 23, wherein said cell-free chloroplast lysate is an extract of a plant cell.

25. The composition of claim 23, wherein said recombination and gene repair activities are provided by a chloroplast-derived enzyme.

26. The composition of claim 13, wherein said oligonucleotide is a duplex mutational vector comprising a contiguous single-stranded self-complementary oligonucleotide having a 3′ end and a 5′ end, wherein said 3′ end and said 5′ end are juxtaposed and wherein at least five contiguous nucleotides are Watson-Crick base paired, the sequence of said oligonucleotide comprising a template for said modified DNA sequence.


SEQUENCE LISTING (14 sequences)

SEQ ID NO: 1 - DNA, 68 bp, Artificial
  chimeric DNA/RNA oligonucleotide Kan4021C
gctattcggctacgactgggcacaattttuugugcccagtcgtagccgaauagcgcgcgttttcgcgc

SEQ ID NO: 2 - DNA, 22 bp, Artificial
  modified single stranded DNA oligonucleotide 3S/25G
ttgtgcccagtagccgaatagc

SEQ ID NO: 3 - DNA, 68 bp, Artificial
  chimeric DNA/RNA oligonucleotide Tet(delta)208T
ttcccacagcattgccagtcactatttttauagugacuggcaatgcugucggaagcgcgttttcgcgc

SEQ ID NO: 4 - DNA, 28 bp, Artificial
  modified single stranded DNA oligonucleotide 3S/28A
catagtgactggcaatgctgtcggaatg

SEQ ID NO: 5 - DNA, 68 bp, Artificial
  chimeric DNA/RNA oligonucleotide SC1
acctgactcctgaggagaagtctgcttttgcagacuucucctcaggagucaggugcgcgttttcgcgc

SEQ ID NO: 6 - DNA, 68 bp, Artificial
  chimeric DNA/RNA oligonucleotide Kan4021G
gctattcggctatgactgggcacaattttuugugcccagtcctagccgaauagcgcgcgttttcgcgc

SEQ ID NO: 7 - DNA, 20 bp, Artificial
  partial DNA sequence of the mutant pK(s)m4021 plasmid
gaggctattcggctaggact

SEQ ID NO: 8 - DNA, 20 bp, Artificial
  partial DNA sequence of the converted pK(s)m4021 plasmid
gaggctattcggctacgact

SEQ ID NO: 9 - DNA, 20 bp, Artificial
  partial DNA sequence of the converted pK(s)m4021 plasmid
gaggctattcggctacgact

SEQ ID NO: 10 - DNA, 19 bp, Artificial
  partial DNA sequence of the converted pK(s)m4021 plasmid
attcggctacgactgggca

SEQ ID NO: 11 - DNA, 20 bp, Artificial
  partial DNA sequence of the converted pK(s)m4021 plasmid
ggctattcggctacgactgg

SEQ ID NO: 12 - DNA, 22 bp, Artificial
  partial DNA sequence of the converted pT(s)(delta)208 plasmid
tccgacagcatgccagtcacta

SEQ ID NO: 13 - DNA, 23 bp, Artificial
  partial DNA sequence of the converted pT(s)(delta)208 plasmid
ttccgacagcattgccagtcact

SEQ ID NO: 14 - DNA, 22 bp, Artificial
  partial DNA sequence of the converted pT(s)(delta)208 plasmid
tccgacagcattgccagtcact